(19) United States
(12) Patent Application Publication
(10) Pub. No.: US 2004/0191833 A1
(43) Pub. Date: Sep. 30, 2004

(54) Selective activation of cellular activities mediated through a common toll-like receptor

(75) Inventors: Jason R. Fink, Eagan, MN (US); Shalley K. Gupta, Woodbury, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/807,934

(22) Filed: Mar. 24, 2004

Related U.S. Application Data
(60) Provisional application No. 60/457,336, filed on Mar. 25, 2003.

(51) Int. Cl. G01N 33/53; C07K 14/00

(52) U.S. Cl. 435/7.1; 514/2; 530/300

(57) ABSTRACT

Methods of identifying compounds that selectively modulate cellular activities mediated by a common TLR are provided. Generally, the methods include providing an assay to detect modulation of a first cellular activity mediated by a TLR; providing an assay to detect modulation of a second cellular activity mediated by the TLR; performing each assay using a test compound; and identifying the test compound as a compound that selectively modulates at least one cellular activity of a plurality of activities mediated by a common TLR if the test compound modulates the first cellular activity to a different extent than it modulates the second TLR-mediated cellular activity. Compounds identified by such methods, pharmaceutical compositions including such compounds, and methods of treating a condition by administering such pharmaceutical compositions to a subject are also provided.

### CROSS-REFERENCE TO RELATED APPLICATION

This application claims priority to U.S. Provisional Patent Application No. 60/457,336, filed Mar. 25, 2003.

### BACKGROUND

Immune response modifiers (“IRMs”) include compounds that possess potent immunomodulating activity including such as, for example, antiviral and antitumor activity. Certain IRMs modulate the production and secretion of cytokines. For example, certain IRM compounds induce the production and secretion of cytokines such as, e.g., Type I interferons, TNF-α, IL-1, IL-6, IL-8, IL-10, IL-12, MIP-1, and/or MCP-1. As another example, certain IRM compounds can inhibit production and secretion of certain T_{H}2 cytokines, such as IL-4 and IL-5. Additionally, some IRM compounds are said to suppress IL-1 and TNF (U.S. Pat. No. 6,518,265).

Certain IRMs are small organic molecules (e.g., molecular weight under about 1000 Daltons, preferably under about 500 Daltons, as opposed to large biological molecules such as proteins, peptides, and the like) such as those disclosed in, for example, U.S. Pat. Nos. 4,689,338; 4,929,624; 4,988,815; 5,037,986; 5,175,296; 5,238,944; 5,266,575; 5,268,376; 5,346,905; 5,352,784; 5,367,076; 5,389,640; 5,395,937; 5,446,153; 5,482,936; 5,693,811; 5,741,908; 5,756,747; 5,939,090; 6,039,969; 6,083,505; 6,110,929; 6,194,425; 6,245,776; 6,331,539; 6,376,669; 6,451,810; 6,525,064; 6,541,485; 6,545,016; 6,545,017; 6,558,951; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; European Patent 0 394 026; U.S. patent Publication Nos. 2002/0016332; 2002/0055517; 2002/0110840; 2003/0133913; 2003/0199538; and 2004/0014779; and International Patent Publication Nos. WO 01/74343; WO 02/46749 WO 02/102377; WO 03/020889; WO 03/043572; WO 03/045391; and WO 03/103584.

Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08595), and certain 3-β-D-ribofuranosylthiazolo[4,5-d]pyrimidine derivatives (such as those described in U.S. Publication No. 2003/0199461).

Other IRMs include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG and are described, for example, in International Patent Publication No. WO 00/75304.

Other IRMs include biological molecules such as aminoalkyl glucosaminide phosphates (AGPs) and are described, for example, in U.S. Pat. Nos. 6,113,918; 6,303,347; 6,525,028; and 6,649,172.

By stimulating certain aspects of the immune system, as well as suppressing other aspects (see, e.g., U.S. Pat. Nos. 6,039,969 and 6,200,592), IRMs may be used to treat many diseases. For example, diseases that may be treated using IRM compounds include, but are not limited to, external genital and perianal warts caused by human papillomavirus, basal cell carcinoma, eczema, essential thrombocythaemia, hepatitis B, multiple sclerosis, neoplastic diseases, psoriasis, rheumatoid arthritis, type I herpes simplex, and type II herpes simplex.

IRM compounds can modulate cell-mediated immunity by inducing secretion of certain immune system regulator molecules such as cytokines. For example, cytokines that are induced by imiquimod or resiquimod include but are not limited to Type I interferons, TNF-α, IL-1, IL-6, IL-8, IL-10, IL-12, MIP-1, and MCP-1. Many IRM compounds share a number of cellular activities, many of which are conserved across species, e.g., induction of co-stimulatory markers, induction of pro-inflammatory cytokines in monocyte/macrophage cells, and activation of transcriptional regulators NF-κB and AP-1.

IRM compounds also can modulate humoral immunity by stimulating antibody production by B cells. Further, various IRMs have been shown to be useful as vaccine adjuvants (see, e.g., U.S. Pat. Nos. 6,083,505 and 6,406,705).

Toll-Like Receptors (TLRs) are a family of immune system receptors that permit cells of the immune system to recognize specific molecular patterns presented by foreign antigens. Activation of the various TLRs induces a range of biological effects including, for example, the secretion of cytokines and antimicrobial peptides. The discovery of different TLRs has led to the identification of TLR-mediated cellular activities that link activation of TLR by a ligand to the biological effects of TLR activation.

### SUMMARY

The present invention provides a method of identifying a compound that selectively modulates at least one cellular activity of a plurality of cellular activities mediated by a common TLR. Generally, the method includes detecting modulation of a first cellular activity mediated by a TLR; detecting modulation of a second cellular activity mediated by the TLR; and identifying the test compound as a compound that selectively modulates at least one cellular activity of a plurality of cellular activities mediated by a common TLR if the test compound modulates the first cellular activity to a different extent than it modulates the second cellular activity.

The present invention also provides compounds thus identified as well as pharmaceutical compositions that include such a compound or a pro-drug of such a compound.

In another aspect, the present invention provides a method of identifying a target compound having a target modulation profile of cellular activities mediated by a common TLR. Generally, the method includes selecting a target modulation profile; determining the modulation profile; and identifying the test compound as a target compound if the modulation profile of the test compound conforms to the target modulation profile.

The present invention also provides compounds thus identified, as well as pharmaceutical compositions that include such a compound or a pro-drug of such a compound.

In another aspect, the present invention provides a method of selectively modulating cells of the immune system. Generally, the method includes identifying a first immune system cell population having a first cellular activity mediated by a TLR, and a second immune system cell population having a second cellular activity mediated by the TLR; selecting a compound that modulates the first cellular activity to a different extent than it modulates the second cellular activity; and contacting cells of the immune system with the selected compound in an amount effective to modulate at least one of the cellular activities, thereby modulating cells of at least one cell population.

Modulating a cellular activity can include detectably increasing the cellular activity or detectably decreasing the cellular activity. Moreover, a cell population may be modulated either in vitro or in vivo.

In another aspect, the present invention provides a method of treating a subject having a condition treatable by selective modulation of cellular activities mediated by a common TLR. Generally, the method includes identifying a target modulation profile of cellular activities mediated by a common TLR effective for treating the condition; selecting a compound having a modulation profile that conforms to the target modulation profile; and administering to the subject an amount of the compound effective for treating the condition.

In certain embodiments, the condition may be an infectious disease such as a viral disease, a fungal disease, a parasitic disease, a bacterial disease, or a prion-mediated disease. In other embodiments, the condition may be a neoplastic condition such as an intraepithelial neoplasm, a pre-cancerous neoplasm, or a cancer.

Various other features and advantages of the present invention should become readily apparent with reference to the following detailed description, examples, and claims. In several places throughout the specification, guidance is provided through lists of examples. In each instance, the recited list serves only as a representative group and should not be interpreted as an exclusive list.

### DETAILED DESCRIPTION OF ILLUSTRATIVE EMBODIMENTS OF THE INVENTION

It has been discovered that certain IRM compounds can selectively modulate one or more cellular activities mediated by a common TLR. That is, certain IRM compounds can modulate one cellular activity mediated by a particular TLR and modulate a second cellular activity mediated by the same TLR to a different extent. The ability to do so may be desirable, for example, for treating certain conditions. For example, one cellular activity may provide a desirable therapeutic or prophylactic benefit, but a second cellular activity may produce an undesirable effect such as, for example, a side effect. If both cellular activities are mediated by the same TLR, one could be forced to choose between, for example, optimizing treatment (i.e., maximizing the desirable benefit) and minimizing side effects. However, an optimal treatment might involve inducing the desirable cellular activity while limiting the undesirable effect. The present invention provides a method of identifying compounds, as well as the compounds themselves, that can, for example, induce a desirable cellular activity and limit induction of an undesirable activity even if both cellular activities are mediated by a common TLR.

In certain cells of the immune system, TLR activation can be associated with activation of the transcription factor NF-κB. NF-κB activation is associated with certain cellular responses to an immunological challenge, such as the production and secretion of pro-inflammatory cytokines such as TNF-α, IL-1, IL-6, IL-8, IL-10, IL-12, MIP-1, and MCP-1. IRM induction of such cellular responses can be demonstrated by measuring activation of the transcription factor NF-κB in response to exposing a cell to an IRM compound (See, e.g., Chuang et al., Journ. of Leuk. Biol, vol. 71, pp. 538-544 (2002), and Hemmi et al., Nature Immunology, vol. 3(2), pp. 196-200 (2002)). Thus, NF-κB-dependent gene expression can be used as a reporter of TLR activation. The extent of NF-κB activation, however, does not necessarily correlate with the extent of the downstream cellular response because the downstream cellular response may be modulated by one or more additional factors.

Induction of certain NF-κB-independent cellular pathways also can be useful as reporters of TLR activation. For example, IFN-α is a cytokine whose induction is NF-κB-independent. IFN-α is secreted by such immune system cells as T lymphocytes, macrophages, plasmacytoid monocytes, dendritic cells, and natural killer cells. IFN-α is involved in regulating a host's innate and adaptive immune responses to an immunological challenge, perhaps by providing a link between the two responses [Brassard et al., Journal of Leukocyte Biology 71:565-581 (2002)]. The innate immune response can include the cell-mediated response of natural killer (NK) cells to a non-self (e.g., neoplastic) or foreign (e.g., viral) antigen. IFN-α also may indirectly regulate the balance between T_{H}1 and T_{H}2 cell populations and, therefore, the innate and adaptive immune responses.

Induction of NF-κB-dependent gene expression and induction of IFN-α production each can be TLR-mediated. Moreover, both cellular activities may be mediated by a single TLR, for example, TLR7.

As used herein, “cellular activities mediated by a common TLR” refers to distinct cellular activities whose activity is regulated by the same TLR and does not in any way refer to the total number of different TLRs that may mediate a particular cellular activity. For example, each of NF-κB activation and IFN-α induction can be mediated through TLR7. Each also may be mediated by one or more additional TLRs. Because each can be mediated by TLR7, NF-κB activation and IFN-α induction are considered to be cellular activities mediated by a common TLR.

In some cases, the selective modulation involves modulating one TLR-mediated cellular activity, but not detectably modulating another TLR-mediated cellular activity.

In other cases, selective modulation involves modulating one TLR-mediated cellular activity in a manner or to an extent that differs from the manner or extent to which another TLR-mediated cellular activity is modulated.

Accordingly, the present invention provides methods of identifying compounds that selectively modulate cellular activities mediated by a common TLR, the compounds thus identified, and pharmaceutical compositions including such compounds; methods of identifying compounds having a particular activity modulation profile for cellular activities mediated by a common TLR, the compounds thus identified, and pharmaceutical compositions including such compounds; methods of selectively modulating certain populations of immune cells; and methods of treating a subject by administering to the subject a compound that selectively modulates at least one cellular activity of a plurality of activities modulated by a common TLR.

For purposes of this invention, the following terms shall have the meanings set forth as follows:

“Activate” and variations thereof refer to any measurable increase in cellular activity.

“Agonist” refers to a compound that can combine with a receptor (e.g., a TLR) to produce a cellular activity. An agonist may be a ligand that directly binds to the receptor. Alternatively, an agonist may combine with a receptor indirectly by, for example, (a) forming a complex with another molecule that directly binds to the receptor, or (b) otherwise results in the modification of another compound so that the other compound directly binds to the receptor. An agonist may be referred to as an agonist of a particular TLR (e.g., a TLR7 agonist) or a particular combination of TLRs (e.g., a TLR ⅞ agonist—an agonist of both TLR7 and TLR8).

“Cellular activity” refers to a biological activity (e.g., cytokine production) that results from an agonist-receptor interaction.

“Induce” and variations thereof refer to any measurable increase in cellular activity. For example, induction of a particular cytokine refers to an increase in the production of the cytokine. As another example, induction of a nucleotide sequence refers to an increase in transcription of (for, e.g., a coding sequence) or from (for, e.g., a regulatory sequence such as a promoter) the nucleotide sequence.

“Inhibit” and variations thereof refer to any measurable reduction of cellular activity. For example, inhibition of a particular cytokine refers to a decrease in production of the cytokine. As another example, inhibition of a nucleotide sequence refers to a decrease in transcription of (for, e.g., a coding sequence) or from (for, e.g., a regulatory sequence such as a promoter) the nucleotide sequence. “Inhibit” or “inhibition” may be referred to as a percentage of a normal level of activity.

“IRM compound” refers to a compound that alters the level of one or more immune regulatory molecules (e.g., cytokines, co-stimulatory markers, or maturation markers) when administered to an IRM-responsive cell. Representative IRM compounds include the small organic molecules, purine derivatives, small heterocyclic compounds, amide derivatives, oligonucleotide sequences, and aminoalkyl glucosaminide phosphates described above.

“Modulate” and variations thereof refer to a substantial increase or decrease in biological activity. A substantial increase or decrease in biological activity is an increase or decrease beyond a desired threshold increase or decrease in the biological activity.

“Modulation profile” refers to a set of cellular activities mediated by a common TLR and the extent to which each of the cellular activities in the set is or can be modulated using an IRM compound. A target modulation profile refers to a particular desired profile of cellular activities mediated by a common TLR, i.e., a theoretical or idealized modulation profile, such as for a target compound to be identified in a screening assay, or for a compound that would modulate biological activity of immune cells in a particular manner. The modulation profile of a given compound refers to the observed profile of cellular activities mediated by a common TLR that are modulated by the given compound and the extent to which each activity is modulated. The observed modulation profile may be compiled from a single source or multiple sources and may be derived from, for example, experimental assay results, clinical or anecdotal observations, or any other suitable source.

“Prodrug” refers to a derivative of a drug molecule that requires a chemical or enzymatic biotransformation in order to release the active parent drug in the body.

“Selective” and variations thereof refer to having a differential or a non-general impact on biological activity. A compound that selectively modulates cellular activities mediated through a common TLR may be termed an “activity-selective” compound.

“TLR expression profile” refers to the identity of the TLRs expressed by a given cell. The TLR expression profile of a given cell may include the set of TLRs naturally expressed by the given cell type. Alternatively, the TLR expression profile of a genetically modified cell may include more or fewer TLRs than cell would naturally express if it had not been genetically modified.

“TLR-mediated” refers to a biological or biochemical activity that results, directly or indirectly, from TLR function. A particular biological or biochemical activity may be referred to as mediated by a particular TLR (e.g., “TLR7-mediated”).

In one aspect, the present invention provides methods of identifying a compound that selectively modulates at least one cellular activity among a plurality of cellular activities mediated by a common TLR. Generally, the methods include providing an assay to detect modulation of a first cellular activity mediated by a TLR; providing an assay to detect modulation of a second cellular activity mediated by the TLR; performing each assay using a test compound; and identifying the test compound as a compound that selectively modulates at least one cellular activity of a plurality of activities mediated by a common TLR if the test compound modulates the first cellular activity to a different extent than it modulates the second TLR-mediated cellular activity.

The method may detect modulation of the TLR-mediated cellular activity by detecting an increase in a TLR-mediated cellular activity, a decrease in a TLR-mediated cellular activity, or both. For example, in some embodiments, the assays selected for the method can include an assay that detects induction of, for example, a first TLR7-mediated cellular activity, and a second assay that detects induction of, for example, a second TLR7-mediated cellular activity. Such a method could identify compounds that either: (a) induce both the first TLR7-mediated cellular activity and the second TLR7-mediated cellular activity, but to varying degrees, or (b) induce one of the TLR7-mediated cellular activities but do not induce the other TLR7-mediated cellular activity. Additionally or alternatively, the method might include one or more assays that detect inhibition of a TLR-mediated cellular activity.

Standard techniques are available to one of ordinary skill in the art for the design and performance of assays that can detect induction and/or inhibition of a cellular activity mediated by any TLR. Suitable techniques are described, for example, in U.S. patent Publication No. 2004/0014779 A1; U.S. patent application Ser. No. 10/732,563, filed Dec. 10, 2003; U.S. patent application Ser. No. 10/732,796, filed Dec. 10, 2003; and U.S. patent application Ser. No. 10/777,310, filed Feb. 12, 2004.

Unless otherwise indicated, an increase or a decrease in cellular activity refers to an increase or decrease in a particular cellular activity compared to that observed in an appropriate control. An assay may or may not be performed in conjunction with the appropriate control. With experience, one skilled in the art may develop sufficient familiarity with a particular assay (e.g., the range of values observed in an appropriate control under specific assay conditions) that performing a control may not always be necessary to determine whether a compound modulates the TLR-mediated cellular activity in a particular assay.

The precise extent to which a TLR-mediated cellular activity is increased or decreased before it is considered substantial and, therefore, modulated for purposes of the invention may vary according to factors known in the art. Such factors may include, for example, the cellular activity observed as the endpoint of the assay, the concentration of the TLR agonist, the method used to measure or detect the endpoint of the assay, the signal-to-noise ratio of the assay, the precision of the assay, and the nature of different assays used to detect modulation of different TLR-mediated cellular activities. Accordingly it is not practical to set forth generally the threshold increase of TLR-mediated cellular activity required to identify a compound as modulating a particular TLR-mediated cellular activity for all possible assays. Those of ordinary skill in the art, however, can readily determine the appropriate threshold with due consideration of such factors.

In some embodiments, for example, the threshold at which the change in cellular activity is considered “substantial” and, therefore, modulated may be at least a two-fold when a TLR agonist is provided at a given concentration. In other embodiments, the threshold at which the change in cellular activity is considered substantial and, therefore, modulated may be at least three-fold. In still other embodiments, the threshold change may be at least five-fold. An increase or decrease in a TLR-mediated cellular activity that fails to meet the threshold change may be considered to be insubstantial (i.e., not substantially changed) and, therefore, not modulated for purposes of the invention. Thus, a compound may be considered selective between two TLR-mediated activities if, for example, the compound increases each cellular activity mediated through a common TLR with respect to a control, but increases one cellular activity to an extent greater than the threshold (i.e., modulated) and increases a second cellular activity to an extent less than the threshold necessary to be considered substantial (i.e., not modulated).

Cells used to practice the methods of the invention may be any cells that express one or more TLRs and permit detection of TLR-mediated biological activity. In some cases, the cells may naturally express one or more TLRs. Cells that naturally express one or more TLRs include but are not limited to primary immune cells such as monocytes, macrophages, Langerhans cells, dendritic cells, Natural Killer cells, polymorphonuclear cells (e.g., neutrophils, basophils, or eosinophils), B lymphocytes, T lymphocytes, and cells derived from any of the foregoing. In some embodiments, the cells may be genetically modified to increase their expression of one or more TLRs. Some genetically modified cells may be derived from host cells that naturally express one or more TLRs, but have been modified to increase expression of one or more TLRs or increase the number of TLRs expressed by the genetically modified cell. Other genetically modified cells may be derived from host cells that lack detectable TLR activity, so that any detectable TLR-mediated biological activity can be attributed to the one or more TLRs introduced into the cell by the genetic modification.

Some assays suitable for use in the methods of the present invention include detecting expression and/or production of one or more cytokines, chemokines, co-stimulatory markers, or proliferation/maturation markers. Such induction may be detected, for example, by detecting an increase in the presence of one or more such molecules in cell culture, either in the culture medium or sequestered within cells of the culture. Alternatively, some assays suitable for methods according to the present invention include detecting modulation of one or more TLR-mediated cellular activities that occur in vivo. Suitable assays may detect, for example, cell maturation—which may require ex vivo histological examination of cells that matured in vivo—or cytokine production.

In some embodiments, the TLR-mediated cellular activity may include production of at least one cytokine including such as, for example, TNF-α, a Type I interferon (e.g., IFN-α, IFN-β, IFN-ω, etc.), IFN-γ, IL-1, IL-6, IL-8, IL-10, IL-12, MIP-1, MCP-1, or any combination thereof. In other embodiments, the TLR-mediated cellular activity may include production of one or more co-stimulatory markers (e.g., CD40, CD80, CD86 etc.), an intercellular adhesion molecule (ICAM, e.g., ICAM-1, ICAM-2, I-CAM-3, etc.), or a proliferation/maturation marker such as, for example, CD83 or CCR7.

Alternatively, TLR-mediated cellular activity may be detected by detecting induction of gene transcription from a promoter that controls expression of one or more cytokines, chemokines, co-stimulatory markers, or maturation markers. For example, an assay may be designed to detect TLR-mediated activation of a promoter such as the NF-κB promoter or the IFN-α1 promoter. As noted above, in some embodiments, detecting TLR-mediated activation of these promoters may include detection of the molecule produced from the induced gene. Alternatively, some assays may be designed so that a reporter gene is operably linked to a TLR-induced promoter—e.g., the NF-κB promoter or the IFN-α1 promoter—so that TLR-mediated induction of the promoter may be readily detected. Many gene expression reporter constructs are commercially available. In one embodiment, a luciferase reporter system may be operably linked to a TLR-inducible promoter such as the NF-κB promoter or the IFN-α1 promoter, so that TLR-mediated induction of the promoter may be detected by detecting the resulting luciferase signal.

In one embodiment, exemplified in Example 1, selective modulation of TLR7-mediated NF-κB activation and TLR7-mediated IFN-α1 induction were assayed. NF-κB activation was measured in genetically modified HEK293 cells (H-TLR7) by detecting TLR7-mediated NF-κB-dependent transcription of a luciferase reporter. IFN-α1 induction was measured in genetically modified Namalwa cells (N-TLR7) by detecting TLR-mediated IFN-α1-induced transcription of a luciferase reporter. Results are shown in Table 2 and are expressed as the fold increase in TLR7-mediated luciferase signal—(H-TLR7/H-vector) and (N-TLR7/N-vector), respectively—normalized to a control in which the cells were treated with vehicle that contained no IRM compound. In this assay, a compound was considered to induce the TLR7-mediated cellular activity if the compound generated at least a two-fold increase in the luciferase signal compared to the vector control.

The assay identified compounds that: (1) selectively modulated TLR7-mediated NF-κB-dependent gene expression, (2) selectively modulated TLR7-mediated IFN-α1 induction, and (3) modulated both TLR7-mediated NF-κB-dependent gene expression and TLR7-mediated IFN-α1 induction.

Compounds listed in Table 1 are compounds, in addition to some of the compounds shown in Example 1 (Table 3), that have been identified as activity-selective compounds - in this case, compounds that, in the assay of Example 1, modulate TLR7-mediated IFN-α1 induction, but do not modulate TLR7-mediated NF-κB-dependent gene expression.

TABLE 1

| Compound Name | Reference |
|---|---|
| N-{4-[4-amino-2-(2-methoxyethyl)-1H-imidazo[4,5- | US 2003/ |
| c]quinolin-1-yl]butyl}quinoline-3-carboxamide | 0144283 |
| | Example 182 |
| N-[3-(4-amino-2-butyl-1H-imidazo[4,5-c]quinolin-1- | U.S. 6,573,273 |
| yl)propyl]morpholine-4-carboxamide | Example 151 |
| N-[4-(4-amino-2-propyl-1H-imidazo[4,5-c]quinolin-1- | U.S. 6,573,273 |
| yl)butyl]-N′-phenylurea | Example 160 |
| 2-butyl-1-[3-(methylsulfonyl)propyl]-1H-imidazo[4,5- | U.S. 6,664,264 |
| c]quinolin-4-amine | Example 19 |
| N-(2-{2-[4-amino-2-(2-methoxyethyl)-1H-imidazo[4,5- | U.S. 6,656,938 |
| c]quinolin-1-yl)ethoxy}ethyl)-N′-phenylurea | Example 1 |
| N-(2-{2-[4-amino-2-(methoxyethyl)-6,7,8,9-tetrahydro- | U.S. 6,656,938 |
| 1H-imidazo[4,5-c]quinolin-1-yl]ethoxy}ethyl)-N′- | Example 2 |
| phenylurea | |
| 2-ethyl-1-[2-(methylsulfonyl)ethyl]-1H-imidazo[4,5- | U.S. 6,667,312 |
| c]quinolin-4-amine | Example 35 |
| N-(2-{2-[4-amino-2-(ethoxymethyl)-1H-imidazo[4,5- | U.S. 6,660,735 |
| c]quinolin-1-yl]ethoxy}ethyl)-N′-phenylurea | Example 53 |

The methods of the present invention can include modulating cellular activity mediated by any TLR. The structural genes of ten human TLRs have been cloned and sequenced. Thus, the structural gene of any one of the ten human TLRs may be introduced into a host cell to provide a genetically modified cell line for use in an assay in a method according to the present invention. In some embodiments, the structural gene of a particular TLR may be cloned into a cell line such as HEK293 cells, Namalwa cells, mouse RAW cells, or fibroblasts. HEK293 cells and Namalwa cells genetically modified in this way may be used to detect modulation of cellular activities mediated by the cloned TLR, as described above.

In certain embodiments, the assays may include one or more appropriate controls to ensure that the assays are performing properly. However, one may accumulate sufficient experience and familiarity with a given assay or the behavior of certain cells in a given assay that appropriate controls may not be required each time the assay is performed.

In some embodiments, the compound can modulate two or more cellular activities mediated by a common TLR, but modulate one activity to a different extent than another activity. For example, a compound may modulate two different cellular activities in an opposite manner, i.e., induce one activity and inhibit the other activity. Alternatively, a compound may modulate two cellular activities in the same manner (i.e., either induce or inhibit both activities), but modulate one activity to a greater extent than the other activity. Alternatively, the compound may detectably modulate one cellular activity, but substantially fail to modulate a second cellular activity to a detectable extent.

The present invention also provides compounds identified by any embodiment of the methods described above. Unless otherwise indicated, reference to a compound throughout this document can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

The methods described above can employ any assay that detects any modulation of any cellular activity mediated by any TLR. Accordingly, the methods described above can be a powerful tool for identifying a broad spectrum of compounds that selectively modulate one or more cellular activities out of a plurality of cellular activities mediated by a common TLR. The compounds thus identified may be incorporated into a pharmaceutical composition. Such pharmaceutical compositions are described below in greater detail.

In another aspect, the present invention provides methods of identifying a target compound having a particular modulation profile for cellular activities mediated by a common TLR. Generally, the methods include selecting a target modulation profile; determining the modulation profile; and identifying the test compound as a target compound if the modulation profile of the test compound conforms to the target modulation profile.

As used herein, a modulation profile includes information regarding one or more TLR-mediated cellular activities. In the context of a target modulation profile, the profile may include one or more desired modulated TLR-mediated biological activities. For example, a particular condition may be treated effectively by differentially modulating biological activities mediated by a common TLR such as, for example, TLR7-mediated NF-κB-dependent gene expression and TLR7-mediated NF-κB-independent IFN-α expression. A target modulation profile for treating that condition might include, for example, (a) modulating one TLR7-mediated cellular activity, but not detectably modulating the other activity, (b) modulating two different cellular activities in an opposite manner (i.e., inducing one activity and inhibiting the other activity), or (c) modulating two cellular activities in the same manner (i.e., either inducing or inhibiting both activities), but modulating one activity to greater extent than the other activity.

A target modulation profile may contain as much or as little information as is known and/or required to provide a desired result. In some cases, the relevant portion of a target modulation profile may include one or more cellular activities mediated by a common TLR (e.g., TLR7) without regard to any other cellular activity mediated by the TLR or any cellular activities mediated by any other TLR. This may be so because of certain factors relating to the condition to be treated or the target cell population whose biological activity is intended to be modulated. Such factors include but are not limited to the identity of TLRs expressed by target cells; the relative levels of expression of the TLRs expressed by the target cells; the presence or absence of additional factors that might also modulate one or more of the cellular activities; the location of the target cells—in vitro, in vivo, and if in vivo, the tissue or organ in which the target cells are located; and, if in vivo, the general state of the subject's immune system (e.g., suppressed, compromised, stimulated).

The modulation profile of a test compound may be determined in any suitable manner. One method of determining the modulation profile of a compound is to perform one or more assays such as the assays described in detail above to determine whether a test compound detectably modulates the biological activity mediated by a particular TLR. Alternatively, certain compounds are already known to be agonists of one or more TLRs, and the biological effects of contacting immune cells with such compounds also may be known. In some cases, at least a portion of a modulation profile of a test compound may be derived from clinical or anecdotal observation of effects of administering the compound to a subject when, for example, the observed effects may be correlated to a particular TLR-mediated biological activity.

The modulation profile of a test compound may contain as much or as little information as is desired for comparison with the target modulation profile. The extent of the information desired for the modulation profile of a test compound may depend, at least in part, on a number of factors including but not limited to the factors listed above with respect to determining the target modulation profile.

Identifying a test compound as conforming to a particular target modulation profile involves comparing the modulation profile of the test compound with the target modulation profile. In some cases, the target modulation profile and the modulation profile of the test compound may be substantially identical or nearly so. In such cases, the test compound can be readily identified as conforming to the target modulation profile.

In certain cases in which the target modulation profile and the modulation profile of the test compound differ to some extent, the test compound may still be identified as conforming to the desired modulation profile. For example, the test compound might modulate a particular TLR-mediated cellular activity that, for the purposes of the target modulation profile, has little if any relevance. Alternatively, in some cases, the target modulation profile can include one or more TLR-mediated cellular activities that are not detectably modulated by a test compound. Different portions of the target modulation profile may be deemed to be of primary and secondary importance, so that a test compound may be identified as conforming to the target modulation profile if the modulation profile of the test compound includes the primary modulation activity, even if it does not include the secondary modulation activity of the target modulation profile. For example, a target modulation profile may include a primary modulation activity of inducing IFN-α1 expression and a secondary modulating activity of inhibiting NF-κB-dependent gene expression. A test compound that adequately induces IFN-α1 expression but, for example, does not modulate NF-κB-dependent gene expression may, in certain circumstances, be considered to conform to the target modulation profile. One of skill in the art, taking all relevant factors into consideration, will be able to determine when meeting the primary modulating activity of the target modulation profile is sufficient so that the modulation profile of the test compound conforms to the target modulation profile even if the test compound profile does not meet a secondary modulation activity.

The target modulation profile may vary with the specific applications for which compounds identified as conforming to the target modulation profile are to be used. For example, treatment of certain viral infections may benefit from administration of a compound that selectively induces TLR7-mediated production of Type I interferons and activates certain antigen presenting cells (APCs).

Alternatively, treatment of certain types of tumors may benefit from using a compound that selectively induces TLR7-mediated NF-κB-dependent gene expression. Such a compound may induce immune system activity localized to the area to which the compound is administered including, for example, induction of IL-12 secretion and a strong inflammatory response.

In another alternative, treatment of some conditions may benefit from administration of a compound that induces both Type I interferon production and NF-κB-dependent gene expression. Such treatment may induce Type I interferon production and IL-12 production, which together synergistically enhance IFN-γ production. IFN-γ production may help facilitate an immune response against malignant cancers including but not limited to melanoma and renal cell carcinoma.

The present invention also provides compounds identified as target compounds according to the method described above. The method described above can employ any suitable target modulation profile for cellular activities modulated by a common TLR, incorporating information relating to the modulation of any number of the cellular activities modulated by any of the TLRs. Accordingly, the methods described above can be a powerful tool for identifying a broad spectrum of compounds that conform to a particular target modulation profile for cellular activities modulated by a common TLR. The compounds thus identified may be incorporated into a pharmaceutical composition. Such pharmaceutical compositions are described in greater detail below.

In another aspect, the present invention provides methods of selectively modulating cells of the immune system. Generally, the methods include identifying a first immune system cell population having a first cellular activity mediated by a TLR, and a second immune system cell population having a second cellular activity mediated by the same TLR; selecting a compound that modulates the first cellular activity to a different extent than it modulates the second cellular activity; and contacting cells of the immune system with the selected compound in an amount effective to modulate at least one of the cellular activities, thereby selectively modulating the cells of at least one cell population.

The immune system includes various populations of cells, each population carrying out one or more finctions that facilitate mounting an effective immune response against an immunological challenge. The various populations of cells populate different areas of the body including but not limited to the blood, skin, bone marrow, thymus, lymphatic system, and interstitial areas. The various populations of immune cells also express the various TLRs to different extents. For example, monocytes express relatively large amounts of TLR2 and TLR4, and also show significant levels of, for example, TLR1 and TLR8 expression. B lymphocytes exhibit relatively high expression of TLR1, TLR6, and TLR10, but also express, for example, TLR7 and TLR9. Plasmacytoid dendritic cells (pDCs) predominantly express TLR9, but also express some TLR1, TLR6, TLR7, and TLR10.

With the discovery that some compounds may modulate at least one biological activity mediated by a TLR, but not modulate another activity mediated by the same TLR, the present invention provides means by which one can selectively modulate cells of the immune system. The selective modulations may take the form of modulating one TLR-mediated cellular activity or population of immune cells while leaving the activity of another cellular activity mediated by the same TLR or another population of immune cells substantially unmodulated (i.e., qualitative or “on-off” modulation). Alternatively, the selective modulation may involve modulating two or more biological activities modulated by a common TLR or two or more populations of immune cells to varying degrees (i.e., quantitative modulation).

In certain embodiments, the methods of the present invention include determining the TLR expression profile of the cells of each cell population. A TLR expression profile may be determined by any suitable method including but not limited to detection of TLR expression such as by PCR analysis, pulse-chase analysis of TLR protein synthesis, and labeling TLRs using TLR-specific antibodies for analyses such as, but not limited to, immunohistochemistry, Western blots, or flow cytometry.

The selective modulation of immune cells may include detectably activating or inducing the cells or detectably inhibiting the cells. The cells of the immune system may be selectively modulated either in vitro or in vivo. In vitro selective modulation may include collecting a sample of immune cells from a subject, culturing the collected immune cells in vitro, and adding the selected compound to the cell culture. The sample of immune cells collected from the subject may be a heterogeneous sample of cells, i.e., the sample may include cells of more than one population of immune cells. After the cells have been selectively modulated, the treated cells may be reintroduced into the subject, thereby providing prophylactic or therapeutic treatment. Alternatively, cells selectively modulated in vitro may have diagnostic utility.

In some embodiments, cells selectively modulated in vitro may be genetically modified rather than collected from a subject. Such cells may have utility as experimental tools, such as, for example, further elucidating TLR-mediated biological activity.

In vivo selective modulation may include administering the selected compound to a subject. The selected compound may be administered in any suitable manner including but not limited to topical, injection (e.g., intravenous, subcutaneous, intraperitoneal, intradermal), inhalation, ingestion, transdermal, or transmucosal delivery.

The particular amount of the selected compound effective for selectively modulating immune cells in a subject may depend, at least in part, on one or more factors. Such factors include but are not limited to the particular compound being administered, the state of the subject's immune system (e.g., suppressed, compromised, stimulated); the identity and location of the cells being modulated; the route of administering the compound; the TLR expression profile of the cells being modulated; and the desired result (e.g., prophylactic or therapeutic treatment). Accordingly it is not practical to set forth generally the amount that constitutes an effective amount of compound. Those of ordinary skill in the art, however, can readily determine the appropriate amount with due consideration of such factors.

An amount of the selected compound effective to selectively modulate cells of the immune system is an amount sufficient to cause the targeted cell population or populations (e.g., monocytes, macrophages, dendritic cells, B cells, T cells, etc.) to alter at least one TLR-mediated biological activity (e.g., cytokine production).

The precise amount of selected compound effective for selectively modulating immune cells will vary according to factors known in the art but in certain embodiments the amount can be a dose of from about 100 ng/kg to about 50 mg/kg, for example, from about 10 μg/kg to about 5 mg/kg. In other embodiments, the amount may be an amount sufficient to provide a final concentration of from about 0.001 μM to about 100 μM of the selected compound in a suitable solution. The minimum amount of the selected compound may vary, dependent upon the factors described above, but may be, in certain embodiments, 0.001 μM, 0.003 μM , 0.01 μM, 0.03 μM, 0.1 μM, 0.3 μM, 1.0 μM, 3.0 μM, or 10 μM. Similarly, the maximum amount of the selected compound may vary, depending upon the factors described above, but may be, in certain embodiments, 100 μM, 30 μM, 10 μM, 3 μM, 1.0 μM, 0.3 μM, or 0.1 μM.

In some embodiments, the selected compound can be a known IRM compound including the small organic IRM molecules described in detail below, or the purine derivatives, small heterocyclic compounds, amide derivatives, and oligonucleotide sequences described above. Alternatively, the selected compound may be a compound capable of selectively modulating at least one cellular activity of a plurality of cellular activities mediated by a common TLR, identified by any suitable method of identifying such compounds, including some of the methods according to the present invention.

As noted above, a compound that selectively modulates a cellular activity out of a plurality of cellular activities mediated by a common TLR (an “activity-selective” compound) may be incorporated into a pharmaceutical composition. Such compositions may be useful for treatment of conditions treatable by selectively modulating one or more cellular activities out of a plurality of cellular activities mediated by a common TLR.

An activity-selective compound can be administered as the single therapeutic agent in the treatment regimen. Alternatively, an activity-selective compound may be administered in combination with another activity-selective compound or with one or more active agents including additional IRM compounds, immunogens, adjuvants, antivirals, antibiotics, anticancers, etc.

Accordingly, the present invention also provides methods of treating a condition treatable by selective modulation of cellular activities mediated by a common TLR. Generally, the methods include identifying a target modulation profile for cellular activities mediated by a common TLR effective for treatment of the condition; selecting an activity-selective compound having a modulation profile for cellular activities mediated by a common TLR that conforms to the target modulation profile; and administering to the subject an amount of the activity-selective compound effective for treating the condition.

Treating a condition may involve either prophylactic or therapeutic treatment. As used herein, prophylactic treatment refers to treatment initiated before the onset of symptoms or signs of the condition. Thus, prophylactic treatments generally are designed to: reduce the likelihood that the subject receiving the treatment will acquire the condition, reduce the severity of the condition, if acquired, or both. As used herein, therapeutic treatment refers to treatment initiated after the onset of symptoms or signs of a condition. Thus, therapeutic treatments are designed to limit or reduce progression of the condition. In some cases, therapeutic treatments can result in reversal of the condition, even to the point of complete resolution.

Identifying the target modulation prqfile for cellular activities modulated by a common TLR may involve determining which immune system cell population or populations might be well-suited for providing prophylactic or therapeutic treatment of the condition, then determining which TLR-mediated cellular activities of the identified cell populations might be modulated to provide the desired treatment.

The modulation profile for cellular activities modulated by a common TLR of the activity-selective compound may be determined by performing one or more assays designed to detect modulation of TLR-mediated cellular activities. Alternatively, the modulation profile for cellular activities modulated by a common TLR of the IRM compound may be determined by clinical or even anecdotal observation.

Selecting an activity-selective compound having a modulation profile for cellular activities modulated by a common TLR that conforms to the target modulation profile involves the same considerations described above relating to assays for identifying a target compound having a particular modulation profile.

Conditions that may be treated by administering an activity-selective compound include, but are not limited to:

(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picomavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);

(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;

(c) other infectious diseases, such chlamydia, fingal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection; and

(d) neoplastic diseases, such as intraepithelial neoplasias, cervical dysplasia, actinic keratosis, basal cell carcinoma, squamous cell carcinoma, renal cell carcinoma, Kaposi's sarcoma, melanoma, renal cell carcinoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers;

(e) T_{H}2-mediated, atopic diseases, such as atopic dermatitis or eczema, eosinophilia, asthma, allergy, allergic rhinitis, and Ommen's syndrome;

(f) certain autoimmune diseases such as systemic lupus erythematosus, essential thrombocythaemia, multiple sclerosis, discoid lupus, alopecia areata; and

(g) diseases associated with wound repair such as, for example, inhibition of keloid formation and other types of scarring (e.g., enhancing would healing, including chronic wounds).

Additionally, an activity-selective compound may be useful as a vaccine adjuvant for use in conjunction with any material that raises either humoral and/or cell mediated immune response, such as, for example, live viral, bacterial, or parasitic immunogens; inactivated viral, tumor-derived, protozoal, organism-derived, fungal, or bacterial immunogens, toxoids, toxins; self-antigens; polysaccharides; proteins; glycoproteins; peptides; cellular vaccines; DNA vaccines; autologous vaccines; recombinant proteins; glycoproteins; peptides; and the like, for use in connection with, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, yellow fever, and Alzheimer's Disease.

Certain activity-selective compounds may be particularly helpful in individuals having compromised immune function. For example, certain compounds may be used for treating the opportunistic infections and tumors that occur after suppression of cell mediated immunity in, for example, transplant patients, cancer patients and HIV patients.

The activity-selective compound may be provided in any formulation suitable for administration to a subject. Suitable types of formulations are described, for example, in U.S. Pat. No. 5,736,553; U.S. Pat. No. 5,238,944; U.S. Pat. No. 5,939,090; U.S. Pat. No. 6,365,166; U.S. Pat. No. 6,245,776; U.S. Pat. No. 6,486,186; European Patent No. EP 0 394 026; and U.S. patent Publication No. 2003/0199538. The activity-selective compound may be provided in any suitable form including but not limited to a solution, a suspension, an emulsion, or any form of mixture. The activity-selective compound may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives such as, for example, adjuvants, skin penetration enhancers, colorants, fragrances, flavorings, moisturizers, thickeners, and the like.

A formulation may be administered in any suitable manner such as, for example, non-parenterally or parenterally. As used herein, non-parenterally refers to administration through the digestive tract, including by oral ingestion. Parenterally refers to administration other than through the digestive tract such as, for example, intravenously, intramuscularly, transdermally, subcutaneously, transmucosally (e.g., by inhalation), or topically.

In some embodiments, an activity-selective compound can be administered to a subject in a formulation of, for example, from about 0.0001% to about 10% (unless otherwise indicated, all percentages provided herein are weight/weight with respect to the total formulation) to the subject, although in some embodiments the activity-selective compound may be administered using a formulation that provides the activity-selective compound in a concentration outside of this range. In certain embodiments, the method includes administering to a subject a formulation that includes from about 0.01% to about 1% activity-selective compound, for example, a formulation that includes from about 0.1 % to about 0.5% activity-selective compound.

An amount of an activity-selective compound effective for treating a condition is an amount sufficient to provide the desired therapeutic or prophylactic benefit. The precise amount of activity-selective compound for treating a condition will vary according to factors known in the art including but not limited to the condition, the physical and chemical nature of the activity-selective compound, the nature of the carrier, the intended dosing regimen, the state of the subject's immune system (e.g., suppressed, compromised, stimulated), the method of administering the activity-selective compound, and the species to which the formulation is being administered. Accordingly, it is not practical to set forth generally the amount that constitutes an amount of activity-selective compound effective for treating a condition for all possible applications. Those of ordinary skill in the art, however, can readily determine the appropriate amount with due consideration of such factors.

In some embodiments, the methods of the present invention include administering sufficient activity-selective compound to provide a dose of, for example, from about 100 ng/kg to about 50 mg/kg to the subject, although in some embodiments the methods may be performed by administering the activity-selective compound in concentrations outside this range. In some of these embodiments, the method includes administering sufficient activity-selective compound to provide a dose of from about 10 μg/kg to about 5 mg/kg to the subject, for example, a dose of from about 100 μg/kg to about 1 mg/kg.

The dosing regimen may depend at least in part on many factors known in the art including but not limited to the condition, the physical and chemical nature of the activity-selective compound, the nature of the carrier, the amount of activity-selective compound being administered, the state of the subject's immune system (e.g., suppressed, compromised, stimulated), the method of administering the activity-selective compound, and the species to which the formulation is being administered. Accordingly it is not practical to set forth generally the dosing regimen effective for treating a condition for all possible applications. Those of ordinary skill in the art, however, can readily determine the appropriate amount with due consideration of such factors.

In some embodiments of the invention, the activity-selective compound may be administered, for example, from a single dose to multiple doses per day, although in some embodiments the methods of the present invention may be performed by administering the activity-selective compound at a frequency outside this range. In certain embodiments, the activity-selective compound may be administered from about once per week to about three times per day such as, for example, administering the activity-selective compound once per day.

The organism treated for a condition may be a plant or animal, particularly a vertebrate. Preferably the organism treated for the disorder is a mammal, such as, but not limited to, human, rodent, dog, cat, pig, sheep, goat, or cow.

In some embodiments, the selected compound can be a known IRM compound including the small organic IRM molecules described in detail below, or the purine derivatives, small heterocyclic compounds, amide derivatives, and oligonucleotide sequences described above. Alternatively, the selected compound may be a compound capable of selectively modulating at least one TLR-mediated cellular activity, identified by any suitable method of identifying such compounds, including some of the methods according to the present invention.

IRM compounds suitable for use in the invention include compounds having a 2-aminopyridine fused to a five membered nitrogen-containing heterocyclic ring. Such compounds include, for example, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, and 6-, 7-, 8-, or 9-aryl or heteroaryl substituted imidazoquinoline amines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, and thioether substituted tetrahydroimidazoquinoline amines; imidazopyridine amines including but not limited to amide substituted imidazopyridine amines, sulfonamido substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; and 1H-imidazo dimers fused to pyridine amines, quinoline amines, tetrahydroquinoline amines, naphthyridine amines, or tetrahydronaphthyridine amines.

In certain embodiments, the IRM compound may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

As used herein, a substituted imidazoquinoline amine refers to an amide substituted imidazoquinoline amine, a sulfonamide substituted imidazoquinoline amine, a urea substituted imidazoquinoline amine, an aryl ether substituted imidazoquinoline amine, a heterocyclic ether substituted imidazoquinoline amine, an amido ether substituted imidazoquinoline amine, a sulfonamido ether substituted imidazoquinoline amine, a urea substituted imidazoquinoline ether, a thioether substituted imidazoquinoline amine, or a 6-, 7-, 8-, or 9-aryl or heteroaryl substituted imidazoquinoline amine. As used herein, substituted imidazoquinoline amines specifically and expressly exclude 1-(2-methylpropyl)-1H-imidazo[4,5-c]quinolin-4-amine and 4-amino-α,α-dimethyl-2-ethoxymethyl-1H-imidazo[4,5-c]quinolin-1-ethanol.

In some embodiments, the selected compound can be a known IRM compound including the small organic IRM molecules described in detail above, or the purine derivatives, small heterocyclic compounds, amide derivatives, and oligonucleotide sequences described above. Alternatively, the selected compound may be a compound capable of selectively modulating at least one cellular activity of a plurality of cellular activities mediated by a common TLR, identified by any suitable method of identifying such compounds, including some of the methods according to the present invention.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

The IRM compounds used in the Examples provided below are identified in Table 2.

TABLE 2

| Compound | Chemical Name | Reference |
|---|---|---|
| IRM1 | 4-{3-[2-(4-amino-1H-imidazo[4,5-c]quinolin-1- | WO 02/46193 |
| | yl)ethoxy]-1-propynyl}-2-thiophenecarboxaldehyde | Example 20 |
| IRM2 | N-{8-[4-amino-2-(2-methoxyethyl)-1H-imidazo[4,5- | U.S. 6,573,273 |
| | c]quinolin-1-yl]octyl}-N′-phenylurea | Example 148 |
| IRM3 | N-[3-(4-amino-2-butyl-1H-imidazo[4,5-c]quinolin-1- | U.S. Ser. No. |
| | yl)propyl]isoquinoline-3-carboxamide | 10/027,218 |
| | | Example 188 |
| IRM4 | N-{3-[4-amino-2-(2-methoxyethyl)-1H-imidazo[4,5- | U.S. 6,573,273 |
| | c]quinolin-1-yl]-2,2-dimethylpropyl}-N′-phenylurea | Example 169 |
| IRM5 | 2-methyl-1-(2-{[(2E)-3-phenylprop-2-enyl]oxy}ethyl)- | WO 02/46189 |
| | 1H-imidazo[4,5-c]quinolin-4-amine | Example 145 |
| IRM6 | 1-[4-(butylthio)butyl]-2-ethyl-1H-imidazo[4,5-c]quinolin- | U.S. 6,667,312 |
| | 4-amine | Example 48 |
| IRM7 | N-{3-[4-amino-2-(2-methoxyethyl)-1H-imidazo[4,5-c]-1- | U.S. 6,677,349 |
| | yl]propyl}benzenesulfonamide | Example 249 |
| IRM8 | 2-hydroxymethyl-1-(2-methylpropyl)-6,7,8,9-tetrahydro- | U.S. 5,352,784 |
| | 1H-imidazo[4,5-c]quinolin-4-amine | Example 94 |
| IRM9 | 2-ethoxymethyl-1-(3-methylbutyl)-1H-imidazo[4,5- | U.S. 5,389,640 |
| | c]quinolin-4-amine | Example 109 |
| IRM10 | 2-butyl-1-(2-methylpropyl)-1H-imidazo[4,5- | U.S. 6,194,425 |
| | c][1,8]naphthyridin-4-amine | Example 12 |
| IRM11 | N¹-[2-(4-amino-2-butyl-1H-imidazo[4,5-c][1,5] | U.S. 6,194,425 |
| | naphthyridin-1-yl)ethyl]-2-amino-4-methylpentanamide | Example 102 |
| IRM12 | 1-{2-[3-(5-pyrimidinyl)propoxy]ethyl}-1H-imidazo[4,5- | WO 02/46193 |
| | c]quinolin-4-amine | Example 9 |
| IRM13 | N-[4-(4-amino-2-butyl-6,7-dimethyl-1H-imidazo[4,5- | U.S. 6,545,016 |
| | c]pyridin-1-yl)butyl]benzamide | Example 1 |
| IRM14 | N-[4-(4-amino-2-pentyl-1H-imidazo[4,5-c]quinolin-1- | U.S. 6,677,349 |
| | yl)butyl]methanesulfonamide | Example 250 |
| IRM15 | 2-[(2-methoxyethoxy)methyl]-1-(2-phenoxyethyl)-1H- | WO 02/46189 |
| | imidazo[4,5-c]quinolin-4-amine | Example 136 |
| IRM16 | N-[3-(4-amino-2-methyl-1H-imidazo[4,5-c]quinolin-1- | U.S. Ser. No. |
| | yl)propyl]cyclopentanecarboxamide | 10/027218 |
| | | Example 206 |
| IRM17 | N-{2-[2-(4-amino-2-methyl-1H-imidazo[4,5-c]quinolin-1- | U. S. Ser. No. |
| | yl)ethoxy]ethyl}benzamide | 10/165449 |
| | | Example 70 |
| IRM18 | N-{4-[4-amino-2-(ethoxymethyl)-1H-imidazo[4,5- | U.S. |
| | c[quinolin-1-yl)butyl}morpholine-4-carboxamide | 6,541,485^{#} |
| IRM19 | N-{2-[2-(4-amino-2-methyl-1H-imidazo[4,5-c]-1- | U.S. Ser. No. |
| | yl)ethoxy]ethyl}methanesulfonamide | 10/165443 |
| | | Example 54 |
| IRM20 | N-{2-[2-(4-amino-2-ethyl-1H-imidazo[4,5-c]quinolin-1- | U.S. Ser. No. |
| | yl)ethoxy]ethyl}-N′-phenylurea | 10/164816 |
| | | Example 50 |
| IRM21 | 1-[4-(methylsulfonyl)butyl]-2-propyl-1H-imidazo[4,5- | U.S. 6,667,312 |
| | c]quinolin-4-amine | Example 30 |
| IRM22 | 2-(ethoxymethyl)-1-(2-methylpropyl)-1H-imidazo[4,5- | U.S. 5,389,640 |
| | c]quinolin-4-amine | Example 40 |
| IRM23 | 2-butyl-1-[5-(methylsulfonyl)pentyl]-1H-imidazo[4,5- | WO 02/46192 |
| | c]quinolin-4-amine | Example 11 |
| IRM24 | N-{2-[4-amino-2-(2-methoxyethyl)-1H-imidazo[4,5- | WO 00/76518^{#} |
| | c]quinolin-1-yl]ethyl}-N′-sec-butylthiourea | |
| IRM25 | N-{2-[4-amino-2-(2-methoxyethyl)-6,7,8,9-tetrahydro- | U.S. |
| | 1H-imidazo[4,5-c]quinolin-1-yl]-1, | 6,331,539^{#} |
| | dimethylethyl}methanesulfonamide | |

^{#}This compound is not specifically exemplified but can be readily prepared using the synthetic methods disclosed in the cited reference.

Cells

HEK293 cells—immortalized human embryonic kidney cells, available from American Type Culture Collection, Manassas, Va, ATCC No. CRL-1573.

Namalwa cells—Burkitt's Lymphoma lymphoblastoid cells, available from ATCC American Type Culture Collection, Manassas, Va, ATCC No. CRL-1432.

TLR7 Activation in HEK293 cells

HEK293 medium was prepared from 90% Minimum Essential Medium (MEM) with 2 mM L-glutamine and Earle's Balanced Salt Solution (Invitrogen Corp., Rockville, Md.) adjusted to contain 1.5 g/L sodium bicarbonate, 0.1 mM non-essential amino acids, and 1.0 mM sodium pyruvate; 10% heat-inactivated fetal calf serum. HEK293 cells were cultured by incubating cells in HEK293 medium overnight at 37° C, 8% CO₂.

Twenty-four hours before transfection, HEK293 cells were adhered to a 10 cm dish (Corning 430167, Corning Inc., Corning, N.Y.) at 37° C., 8% CO₂. The cells were co-transfected with (1) pIRES (BD Biosciences Clontech, Palo Alto, Calif.) either (a) unmodified (H-vector), or (b) containing an expressible human TLR7 gene (H-TLR7), and (2) NF-kB-luc reporter (Stratagene, La Jolla, Calif.) in a 10:1 ratio with Fugene 6 transfection reagent (Roche Diagnostics Corp., Indianapolis, Ind.) following the manufacturer's instructions. The plates were incubated for 24 hours following transfection and then selected in G-418 (400 μg/mL) for two weeks. The G-418-resistant cells containing either the expressible TLR7 gene or the empty vector were expanded in HEK293 medium supplemented with G-418 for stimulation experiments.

The transfected cells were plated in white opaque 96 well plates (Costar 3917, Corning Inc., Corning, N.Y.) at a concentration of 5×10⁴ cells per well in 100 μL of HEK293 media and incubated at 37° C., 8% CO₂ for 4 hours. The cells were stimulated with 1 μL of IRM compounds at 1 mM in DMSO (final IRM concentration of 10 μM) or 1 μL DMSO as a control. The plates were then incubated an additional 16 hours at 37° C., 5% CO₂. The luciferase signal was read using the LucLite kit (Packard Instrument Co., Meriden, Conn.). Luminescence was measured on an LMAX luminometer (Molecular Devices Corp., Sunnyvale, Calif.).

TLR7 Activation in Namalwa Cells

Unless otherwise indicated, all incubations were performed at 37° C. with 5% CO₂ at 98% humidity.

Culture medium was prepared from complete RPMI 1640 medium (BioSource International, Inc., Camarillo, Calif.). Fetal bovine serum (Atlas Biologicals, Inc., Ft. Collins, Colo.) was added to a final concentration of 7.5% (vol/vol); L-glutamine (BioSource International, Inc.) was added to 5 mM; and sodium pyruvate (BioSource International, Inc.) was added to 1 mM.

Namalwa cells were grown by incubation in culture medium overnight. Cells were harvested by centrifugation in a tabletop centrifuge (1200 RPM for 5 minutes), and then resuspended in phosphate buffered sucrose to a concentration of 1.3×10⁷ cells/mL.

For each transfection, a 750 μL aliquot of the cell suspension was placed in an electroporation cuvette with 4 mm gaps. Each aliquot received transfection DNA: 10 μg of pGL3-Enhancing vector (Promega Corp., Madison, Wis.) containing the human IFN-α1 promoter cloned into the BglII site (pIFN-α1-luc), and 10 μg of pCI-neo mammalian expression vector (Promega Corp.) either (1) unmodified (N-vector) or (2) containing an expressible human TLR7 gene (N-TLR7). The cell and vector mixtures were incubated at room temperature for 5 minutes. The cells were electroporated using a BioRad Gene Pulser (BioRad Laboratories, Hercules, Calif.) set to at 500 μF capacitance and 0.27 volts, then incubated at room temperature for 5 minutes.

The electroporated cells were suspended in 10 mL of culture medium and incubated overnight. Dead cells and debris were removed after 24 hours using a MACS Dead Cell Removal kit (Miltenyi Biotec, Auburn, Calif.). Cells were resuspended in 10 mL of culture medium and incubated for an additional 24 hours.

Transfected cells were selected by adding G418 (Promega Corp., Madison, Wisc.) to a final concentration of 1 mg/mL and incubating the cells for seven days.

The selected transfected cells were counted and resuspended to a concentration of 1×10⁶ cell per mL in culture medium. 100 μl aliquots of cells were placed in the wells of a white-walled, white-bottomed 96-well plate (Corning, Inc. Corning, N.Y.). 1.0 μL of an IRM compound from Table 1 (prepared at 1 mM in 100% DMSO) was added to some cell aliquots so that the final concentration of IRM compound was 10 μM. As a positive control, some cell aliquots were incubated with Sendai virus instead of IRM compound. As a negative control, some cell aliquots were incubated with DMSO without IRM compound. In all cases, the cells were incubated for 18 hours.

The plates were equilibrated to room temperature before 1 volume of reconstituted LucLight Plus (Packard Instruments, Meriden, Conn.) was added to each aliquot of cells. Each well of the plate was read on an LJL Analyst (LJL Biosystems, Inc., Sunnyvale, Calif.) set with a 5 minute dark adapt.

Results are reported in Table 3. The data are expressed as fold increase in TLR7-mediated luciferase signal—(H-TLR7/H-vector) and (N-TLR7/N-vector), respectively—normalized to the DMSO without compound control.

TABLE 3

| Compound | H-TLR7 | N-TLR7 |
|---|---|---|
| IRM1 | 13.2 | 1.6 |
| IRM2 | 4.1 | 1.5 |
| IRM3 | 4.2 | 1.9 |
| IRM4 | 15.0 | 1.6 |
| IRM5 | 3.4 | 1.6 |
| IRM6 | 7.1 | 1.4 |
| IRM7 | 4.7 | 1.6 |
| IRM8 | 0.4 | 3.0 |
| IRM9 | 0.7 | 2.6 |
| IRM10 | 1.1 | 4.4 |
| IRM11 | 0.5 | 2.8 |
| IRM12 | 0.6 | 2.6 |
| IRM13 | 1.0 | 2.6 |
| IRM14 | 0.9 | 3.3 |
| IRM15 | 0.8 | 2.7 |
| IRM16 | 0.9 | 2.8 |
| IRM17 | 0.4 | 3.1 |
| IRM18 | 0.5 | 3.3 |
| IRM19 | 0.9 | 3.3 |
| IRM20 | 0.8 | 3.7 |
| IRM21 | 0.7 | 3.8 |
| IRM22 | 5.5 | 2.2 |
| IRM23 | 9.4 | 2.8 |
| IRM24 | 7.9 | 3.2 |
| IRM25 | 7.6 | 3.0 |

The complete disclosures of the patents, patent documents, and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

What is claimed is:

1. A method of identifying a compound that selectively modulates at least one cellular activity of a plurality of cellular activities mediated by a common TLR, the method comprising:
(1) detecting modulation of a first cellular activity mediated by a TLR;
(2) detecting modulation of a second cellular activity mediated by the TLR; and
(3) identifying the test compound as a compound that selectively modulates at least one cellular activity of a plurality of cellular activities mediated by a common TLR if the test compound modulates the first cellular activity to a different extent than it modulates the second cellular activity.

2. The method of claim 1 wherein the test compound modulates the first cellular activity and does not modulate the second cellular activity.

3. A compound identified according to the method of claim 1.

4. A pharmaceutical composition comprising a compound identified according to the method of claim 1 or a pro-drug thereof.

5. A method of identifying a target compound having a target modulation profile of cellular activities mediated by a common TLR, the method comprising:
(1) selecting a target modulation profile of cellular activities mediated by a common TLR;
(2) determining the modulation profile of cellular activities mediated by a common TLR for a test compound; and
(3) identifying the test compound as a target compound if the modulation profile of the test compound conforms to the target modulation profile.

6. The method of claim 5 wherein the target modulation profile includes one or more TLR-mediated cellular activities that are not detectably modulated by a target compound.

7. The method of claim 5 wherein determining the modulation profile of a test compound comprises performing at least one assay for detecting modulation of a TLR-mediated cellular activity.

8. A compound identified as a target compound according to the method of claim 5.

9. A pharmaceutical composition comprising a target compound identified according to the method of claim 5 or a pro-drug thereof.

10. A method of selectively modulating cells of the immune system, the method comprising:
(1) identifying a first immune system cell population having a first cellular activity mediated by a TLR, and a second immune system cell population having a second cellular activity mediated by the TLR;
(2) selecting a compound that modulates the first cellular activity to a different extent than it modulates the second cellular activity; and
(3) contacting cells of the immune system with the selected compound in an amount effective to modulate at least one of the cellular activities, thereby modulating cells of at least one cell population.

11. The method of claim 10 wherein the method further comprises determining a TLR expression profile of the first cell population and a TLR expression profile of the second cell population.

12. The method of claim 11 wherein the step of selecting a compound comprises comparing the TLR expression profile of the first cell population and the TLR expression profile of the second cell population with a TLR-mediated cellular activity modulation profile for the compound.

13. The method of claim 10 wherein modulating a cellular activity comprises detectably increasing the cellular activity or detectably decreasing the cellular activity.

14. The method of claim 10 wherein the compound modulates the first cellular activity and does not detectably modulate the second cellular activity.

15. The method of claim 10 wherein the compound modulates both cellular activities.

16. The method of claim 10 wherein at least one cell population is modulated in vitro.

17. The method of claim 10 wherein at least one cell population is modulated in vivo.

18. A method of treating a subject having a condition treatable by selective modulation of cellular activities mediated by a common TLR, the method comprising:
(1) identifying a target modulation profile of cellular activities mediated by a common TLR effective for treating the condition;
(2) selecting a compound having a modulation profile of cellular activities mediated by a common TLR that conforms to the target modulation profile; and
(3) administering to the subject an amount of the compound effective for treating the condition.

19. The method of claim 18 wherein the condition is an infectious disease or a neoplastic condition.

20. The method of claim 19 wherein the infectious disease is a viral disease, a fungal disease, a parasitic disease, a bacterial disease, or a prion-mediated disease.

21. The method of claim 19 wherein the neoplastic condition is an intraepithelial neoplasm, a pre-cancerous neoplasm, or a cancer.
